(19) United States
(12) Patent Application Publication
(10) Pub. No.: US 2004/0127814 A1
(43) Pub. Date: Jul. 1, 2004

(54) Biopsy assembly

(76) Inventor: Carlos Negroni, Kalamazoo, MI (US)

Correspondence Address: HOWARD & HOWARD ATTORNEYS, P.C., THE PINEHURST OFFICE CENTER, SUITE #101, 39400 WOODWARD AVENUE, BLOOMFIELD HILLS, MI, 48304-5151, US

(21) Appl. No.: 10/734,954

(22) Filed: Dec. 12, 2003

Related U.S. Application Data
(60) Provisional application No. 60/433,292, filed on Dec. 12, 2002.

(51) Int. Cl. A61B 10/00

(52) U.S. Cl. 600/567

(57) ABSTRACT

An access cannula (34) is supported by a cannula handle (22) and receives an introduction stylet (40) for insertion into a bone (44). An inner cannula (66) is inserted into the access cannula (34) and advanced into the bone (44) to cut and retrieve a specimen (74). The distal end (70) of the inner cannula (66) includes a swaged portion with a non-deformable sidewall (76) having a frustoconical interior surface (78) extending radially inwardly at a predetermined angle from the distal end (70) to a biopsy opening (80) having an annular cutting edge (84). The cutting edge (84) has opposed cusps (88) extending therefrom. A syringe (100) applies a vacuum to the inner cannula (66) for retaining the biopsy specimen in the inner cannula (66) and an obturator (102) removes the biopsy specimen from the inner cannula (66). The access cannula (34) remains in position and creates an open passage for access to the site (46) for performing other procedures.

### REFERENCES TO RELATED APPLICATIONS

This application claims the benefit of U.S. Provisional Patent Application Serial No. 60/433,292, which was filed on Dec. 12, 2002.

### BACKGROUND OF THE INVENTION

1. Field of the Invention

This invention relates to a biopsy device for collecting a biopsy specimen from an anatomical site. More specifically, the subject invention relates to a biopsy device with coaxially positioned cannulas for collecting a biopsy specimen from a bone while simultaneously minimizing trauma to the bone from which the specimen is collected and to tissues adjacent the bone.

2. Description of the Related Art

Numerous assemblies exist in the art for piercing through a bone to harvest a biopsy specimen of bone marrow therefrom. For example, it is known to advance an access cannula having an introduction needle coaxially positioned therein into a bone to establish an access pathway leading to a predetermined site from which a biopsy specimen is collected. Unfortunately, once the pathway has been defined and the access cannula and needle withdrawn, the pathway must be re-established each time an additional instrument is introduced therethrough, and undergoes additional trauma after the specimen is cut from the site and removed therefrom. Repeatedly inserting instruments through the unprotected pathway subjects the tissue surrounding the pathway and harvest site to unnecessary trauma.

Although certain assemblies utilize an access cannulas within which cannulas are coaxially positioned to minimize the need to re-establish the access pathway, such assemblies possess certain limitations. One such assembly utilizes a inner cannula having a distal end defining an interior chamber into which a plurality of threads radially extend. The threads are embedded into the specimen as the specimen is received within the chamber. While the threads may assist in severing the specimen from the remaining tissue at the harvest site and help retain the specimen within the chamber as the cannula is withdrawn, the threads “work” too well, in that the grip the embedded threads place on the specimen makes it difficult to remove from the chamber without compromising the structural integrity of the specimen.

Other assemblies attempt to simplify the process of harvesting a specimen by incorporating a specialized cutting edge on the distal end of the inner cannula. However, to the extent such a cutting edge is capable of penetrating the bone tissue without breaking or otherwise bending and inflicting additional trauma to the site or specimen, that success is offset by the other components of the assembly, which fail to act in concert with the cutting edge to collect an intact biopsy specimen of sufficient size without sacrificing the access pathway to the harvest site.

### SUMMARY OF THE INVENTION AND ADVANTAGES

The subject invention provides a biopsy assembly for collecting a biopsy specimen from a mass of bone. The assembly includes an inner cannula for being coaxially positioned within an access cannula and formed to sever a biopsy specimen from the bone and retain the specimen therein. More specifically, the inner cannula extends from a proximal end to the distal end. A swaged portion extends from the distal end for retaining the biopsy specimen. The swaged portion includes a non-deformable sidewall with a frustoconical interior surface that extends radially inwardly at a predetermined angle from the distal end to the biopsy opening for receiving the biopsy specimen therethrough.

The subject invention also provides a method of collecting a biopsy specimen from a bone in which an introduction stylet is disposed within the access cannula and is then inserted into the bone to establish a harvest site. The stylet is withdrawn, and an inner cannula is inserted within the access cannula. A portion of the inner cannula is swaged to form a non-deformable sidewall having a frustoconical interior surface extending radially inwardly at a predetermined angle from the distal end to converge at a continuous annular cutting edge defining a biopsy opening. The cutting edge severs a biopsy specimen from the harvest site as the cannulas are simultaneously advanced a predetermined distance into the bone. As the method proceeds, the access cannula maintains the tract to the harvest site, which eliminates the need to need to re-establish the tract to the site after the specimen is retrieved. Therefore, once the inner cannula with the specimen therein is removed from the access cannula, the access cannula remains as an open pathway for use in accessing the harvest site to perform other procedures including, but not limited to injecting bone cement into the site. The access and inner cannulas work in concert rather than acting as independent devices in independent steps, thereby reducing the number of steps and minimizing tissue trauma.

Accordingly, the subject invention overcomes the limitations of the related art by providing a biopsy assembly that not only maintains a protected access pathway to the harvest site for permitting additional procedures to be performed, but also utilizes an inner cannula having a unique distal end for severing and removing a specimen in a manner that minimizes trauma to the specimen and harvest site.

### BRIEF DESCRIPTION OF THE SEVERAL VIEWS OF THE DRAWINGS

Other advantages of the present invention will be readily appreciated as the same becomes better understood by reference to the following detailed description when considered in connection with the accompanying drawings wherein:

FIG. 1 is an exploded perspective view of a biopsy assembly according to an embodiment of the present invention;

FIG. 2 is a side view of the access cannula and stylet utilized in the assembly of the present invention inserted initially through the cortex of a bone;

FIG. 3 is a fragmentary cross-sectional view of the stylet cap and cannula handle utilized in the present invention;

FIG. 4 is a fragmentary side view of the distal end of the access cannula and stylet initially positioned in the bone;

FIG. 5 is a fragmentary side view of the access cannula, bone and stylet, with the stylet withdrawn from the access cannula;

FIG. 6 is a side view of the inner cannula and the cannula handle;

FIG. 7 is a fragmentary cross-sectional view of the swaged portion of the inner cannula;

FIG. 8 is a fragmentary perspective view of the distal end and swaged portion of the inner cannula;

FIG. 9 is a perspective view of the tool handle being locked to the access cannula handle;

FIG. 10 is a fragmentary side view of the tool handle positioned within the cannula handle;

FIG. 11 is a side view of the inner cannula inserted fully within the access cannula with the swaged portion extending into the harvest site;

FIG. 12 is a fragmentary side view of the distal and open ends of the inner and access cannulas, respectively, fully extended into the bone marrow;

FIG. 13 is a side view of the access cannula and inner cannula with a syringe coupled to the inner cannula;

FIG. 14 is a side view of the inner cannula and partial view of an obturator inserted therein;

FIG. 15 is a fragmentary cross-sectional view of the swaged portion of the inner cannula cutting into the harvest site;

FIG. 16 is a fragmentary cross-sectional view of the swaged portion of the inner cannula severing a specimen from the harvest site; and

FIG. 17 is a fragmentary cross-sectional view of the swaged portion and fragmentary view of the obturator inserted therethrough and removing the specimen therefrom.

### DETAILED DESCRIPTION OF THE INVENTION

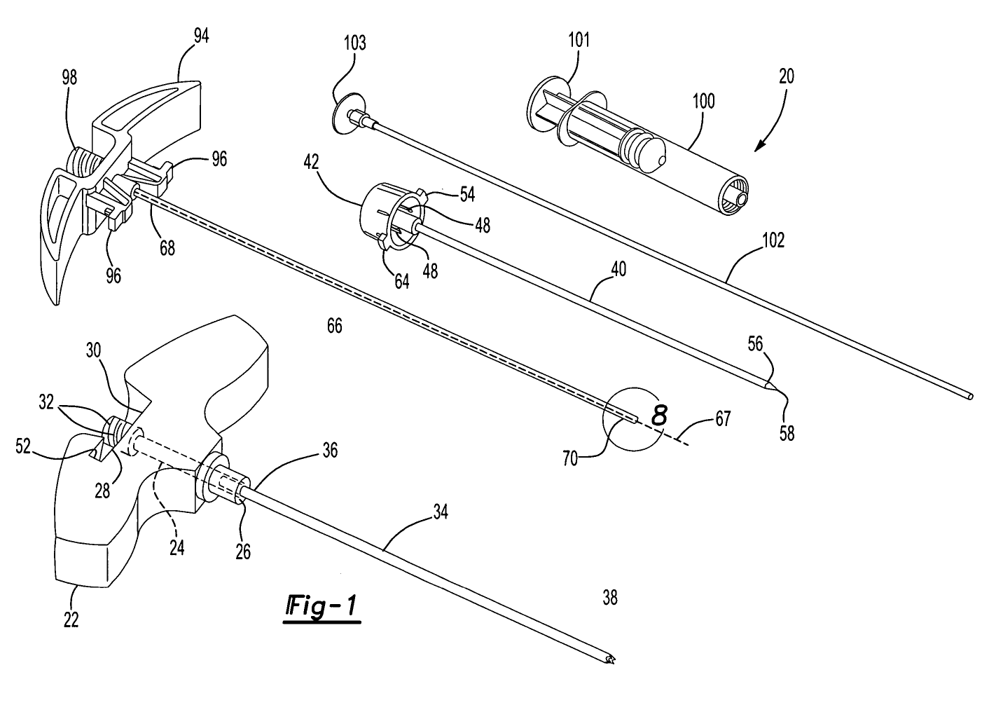

Referring to the Figures, wherein like numerals indicate like or corresponding parts throughout the several views, a biopsy assembly for insertion into a mass of bone to collect a biopsy specimen of tissue is shown generally at 20 in FIG. 1.

The assembly 20 includes a cannula handle 22 with a passageway 24 extending therethrough from a support end 26 to a receiving end 28. The receiving end 28 defines a recess 30, which is disposed about the passageway 24. Male luer threads 32 are disposed in the recess 30 about the receiving end 28. The assembly 20 also includes an access cannula 34 having a proximal end 36 supported in the passageway 24 of the handle 22. The access cannula 34 extends from the proximal end 36 to an open end 38. Although the open end 38 is defined by a toothed cutting edge, any suitable cutting edge may be utilized.

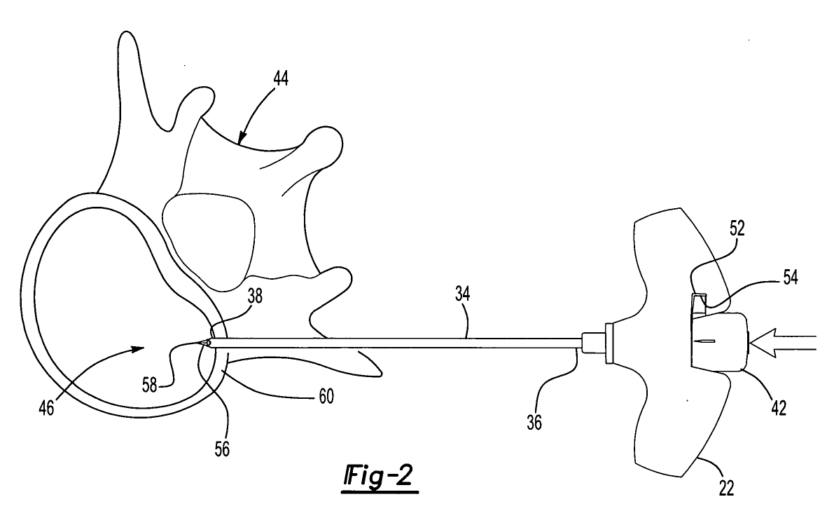

An introduction stylet 40 is also utilized in the assembly 20. A cap 42 supports the stylet 40. As is shown in FIGS. 2 through 4, the stylet 40 is selectively inserted into the passageway 24 and through the access cannula 34 and then used to advance the access cannula 34 into a mass of bone 44 to establish a biopsy harvest site in the bone marrow. The harvest site is shown generally at 46. The cap 42 has opposed fingers 48 that selectively engage the male luer threads 32 of the cannula handle 22 for guiding the cap 42 into proper alignment within the recess 30.

Although the bone 44 shown throughout the Figures is a human vertebrae, the term “bone” as used herein refers to any mass of bone of any living or non-living organism, regardless of whether the mass is in situ and regardless of the origin of the mass of bone.

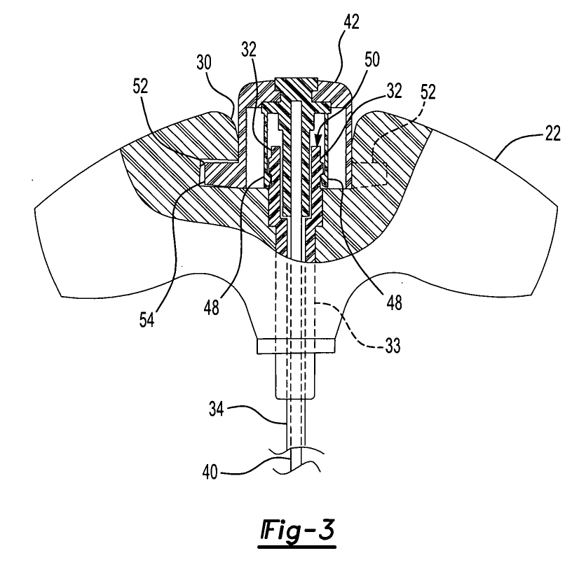

The cannula handle 22 and the cap 42 also present a coacting tongue and groove connection 50 for maintaining the cap 42 and stylet 40 in a stationary position relative to the access cannula 34 and cannula handle 22. The tongue and groove connection 50 engages upon rotation of the cap 42 relative to the handle 22. As is shown in FIG. 3, a plurality of grooves 52 extend radially into the cannula handle 22 within the recess 30, and a plurality of tongues 54 extend radially from the cap 42. The tongues 54 engage the grooves 52 upon rotation of the cap 42 within the recess 30, which in turn interlocks the cap 42 and handle 22. The cannula handle 22 and cap 42 may alternatively be preassembled for use with the stylet 40 and coaxially positioned within the access cannula 34. Although any suitable stylet and cannula may be utilized for accessing the bone 44, the stylet 40 and cannula 34 and respective cap 42 and handle 22 associated therewith are of the type manufactured and sold by Manan Medical Products, Inc.

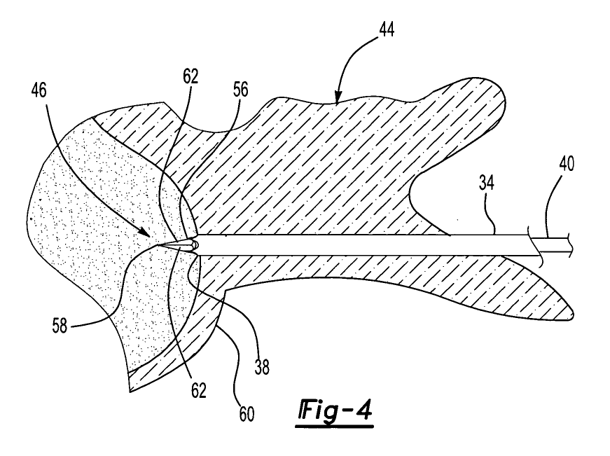

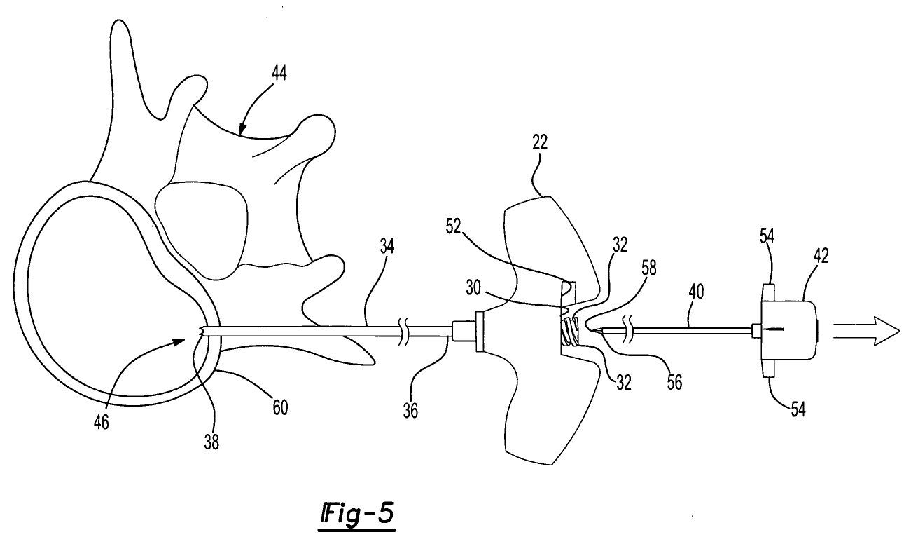

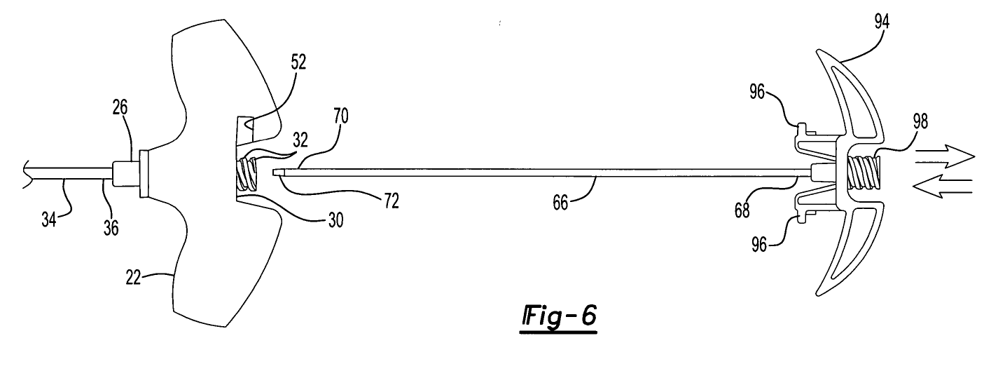

Referring now to FIG. 4, the stylet 40 is inserted into the passageway 24 and through the access cannula 34 to close the open end 38 thereof. The stylet 40 extends to a distal tip 56 that terminates in a sharp point 58. The style 40 is inserted through the passageway 24 of the access cannula 34 until the tip 56 extends through the open end 38. This permits the tip 56 to be introduced into the mass of bone 44 and utilized to advance the stylet 40 and access cannula 34 into the mass 44 to establish the harvest site 46.

Although the distal tip of the stylet 40 may have any configuration suitable for piercing the cortex 60 of the bone 44, the distal tip 56 shown in FIG. 4 includes four beveled facets 62 which converge to define the point 58. The facets 62 cooperate with the point 58 to bore through the bone 44 in response to rotation of the stylet 40 within the access cannula 34.

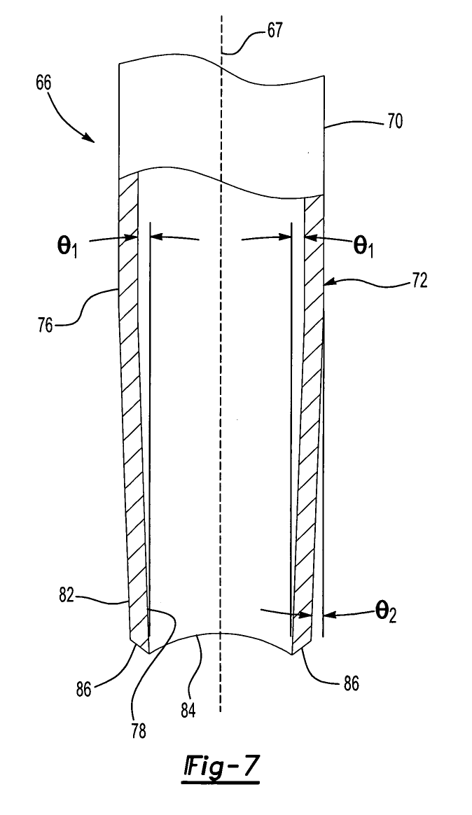

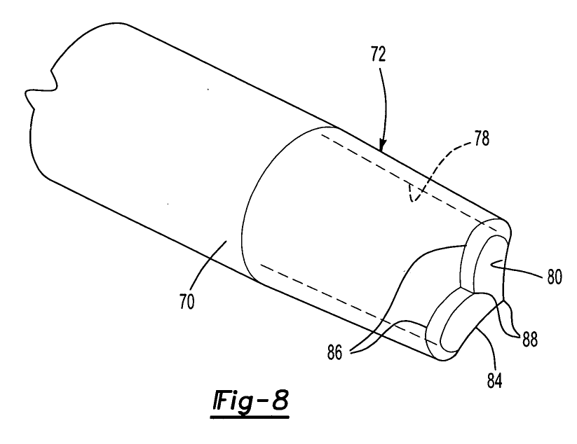

Referring again to FIG. 1, the assembly 20 also includes an inner cannula 66 having a longitudinal axis 67 and extending from a proximal end 68 to a distal end 70. The inner cannula 66 is selectively inserted through the passageway 24 of the cannula handle 22 and into the access cannula 34. As is best shown in FIGS. 7 and 8, a swaged portion 72 extends from the distal end 70. The portion 72 is uniquely designed to retain a biopsy specimen 74 therein. In particular, the swaged portion 72 features a non-deformable sidewall 76 having a frustoconical interior surface 78 that extends radially inwardly at a predetermined angle “θ₁” from the distal end 70 to a biopsy opening 80. The opening 80 receives the biopsy specimen 74 therethrough.

The sidewall 76 also has an exterior surface 82, and the biopsy opening 80 has an annular cutting edge 84 that radially extends from the longitudinal axis 67. The exterior surface 82 extends radially inwardly at a predetermined angle “θ₂” from the distal end 70 to the cutting edge 84. Beveled facets 86 extend between the exterior surface 82 and the cutting edge 84, which in turn renders the cutting edge 84 sharp. The facets 86 also converge at the cutting edge 84 to define a pair of opposed cusps 88.

The manner in which the cutting edge 84 is rendered sharp facilitates cutting into the cancellous tissue in the bone mass 44 and through the trabeculae of the bone marrow at the harvest site 46. In particular, an increased amount of pressure may be applied at the cutting edge 84 and to the site 46 for achieving a cleaner, more accurate cut into the bone 44, while simultaneously minimizing trauma to tissue surrounding the harvest site 46 and preserving the structural integrity of the specimen 74 severed therefrom. Furthermore, the angle at which each facet 86 extends from the cutting edge 84 results in cusps 88 which are sufficiently sharp to pierce the cortex 60, yet have a profile shallow enough to reduce the likelihood of breaking and becoming embedded in the tissue at the harvest site 46.

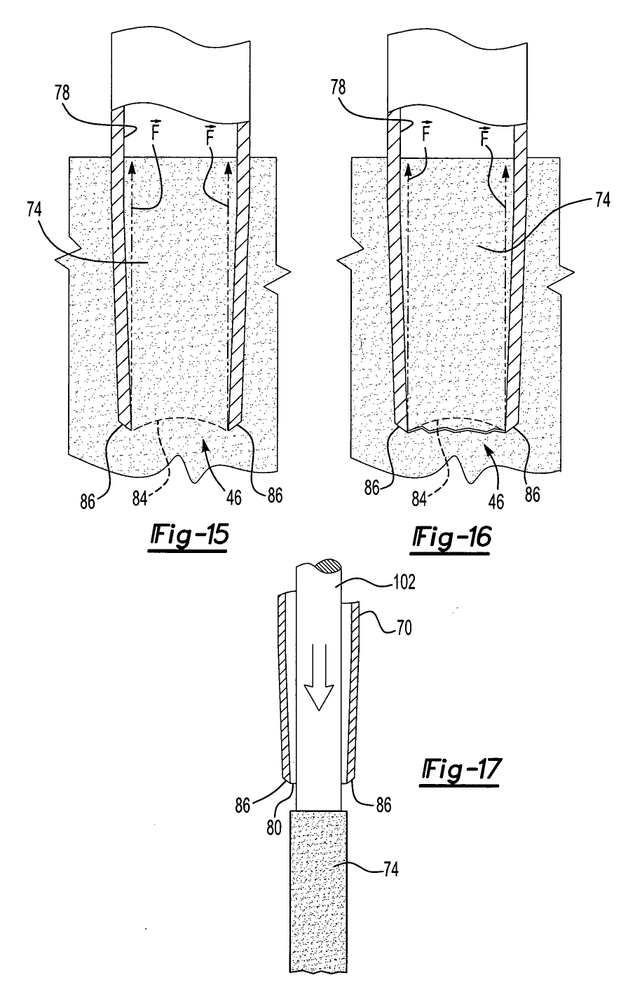

Although the interior surface 78 of the swaged portion 72 may extend at any angle between the distal end 70 and the biopsy opening 80, the predetermined angle “θ₁” at which the interior surface 70 extends from the longitudinal axis 67 of the inner cannula 66 is 2°. The exterior surface 82 may extend at any suitable angle between from the distal end 70 to the cutting edge 84; however, like the interior surface 70, the exterior surface 82 extends at a 2° relative to the longitudinal axis 67 of the inner cannula 66. As is shown in FIGS. 15 and 16, the angle “θ₁” at which the interior surface 70 extends relative to the longitudinal axis 67 creates an increased axial load, or force, “F” on the specimen 74 in the direction shown when the inner cannula 66 is withdrawn from the harvest site 46. This facilitates separation of the specimen 74 from the site 46. Once the specimen 74 has been separated from the site 46, the angle “θ₁” of the interior surface 70 further serves to retain the specimen 74 within the swaged portion 72 until the specimen 74 is removed therefrom through the biopsy opening 80.

The frustoconical interior surface 78 and exterior surface 82 are formed by swaging the portion 72. Swaging is a process in which a stamp, die or other suitable implement is used in conjunction with a hammer or other suitable tool to bend or shape cold metal. Although the portion 72 is formed by swaging, the portion 72 may be formed using any suitable alternative process.

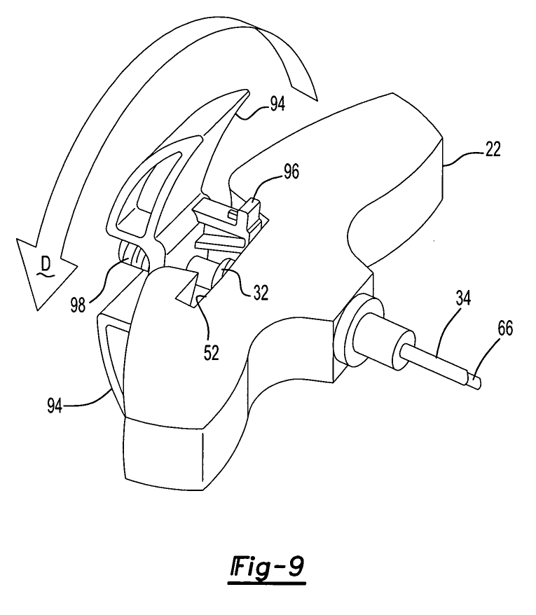

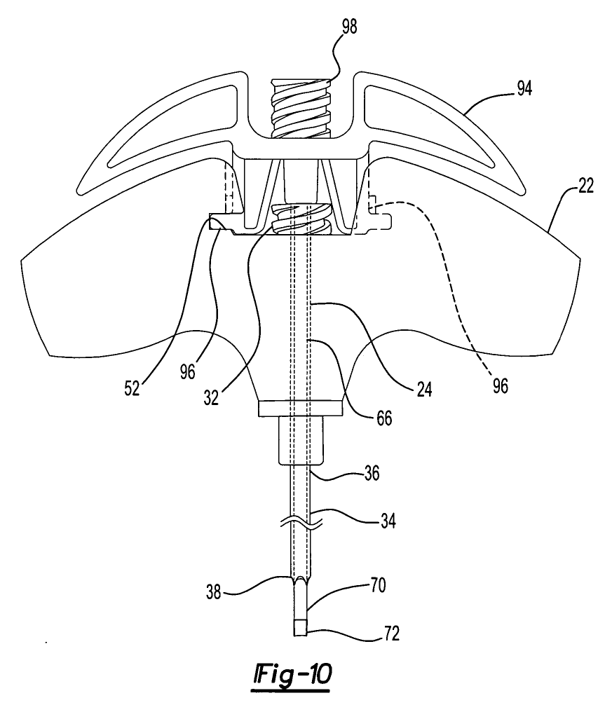

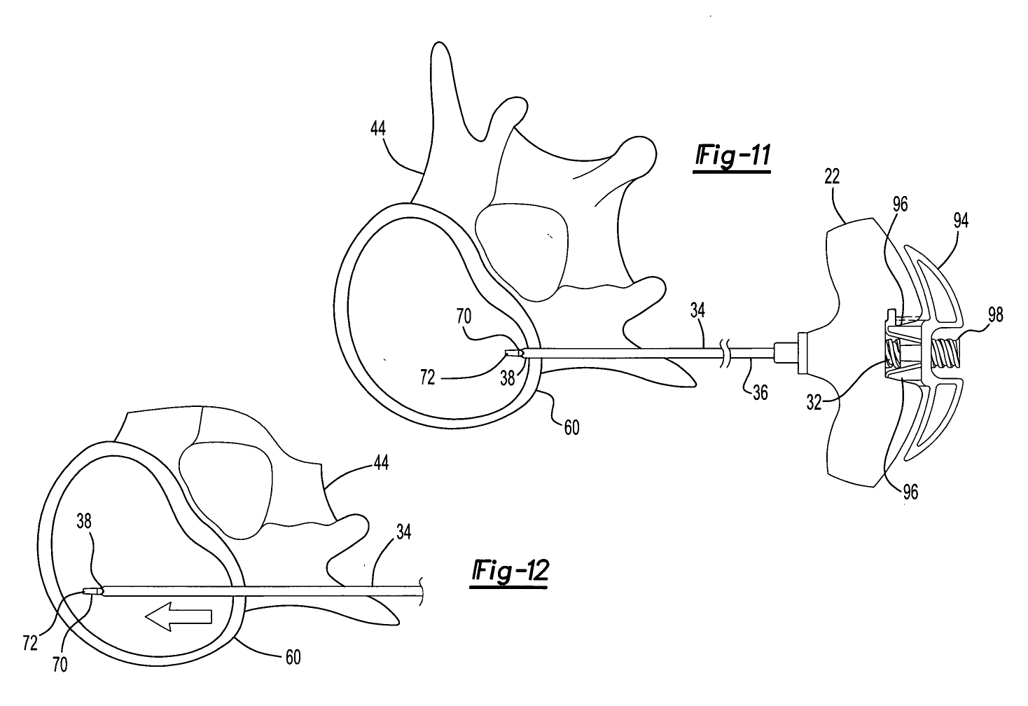

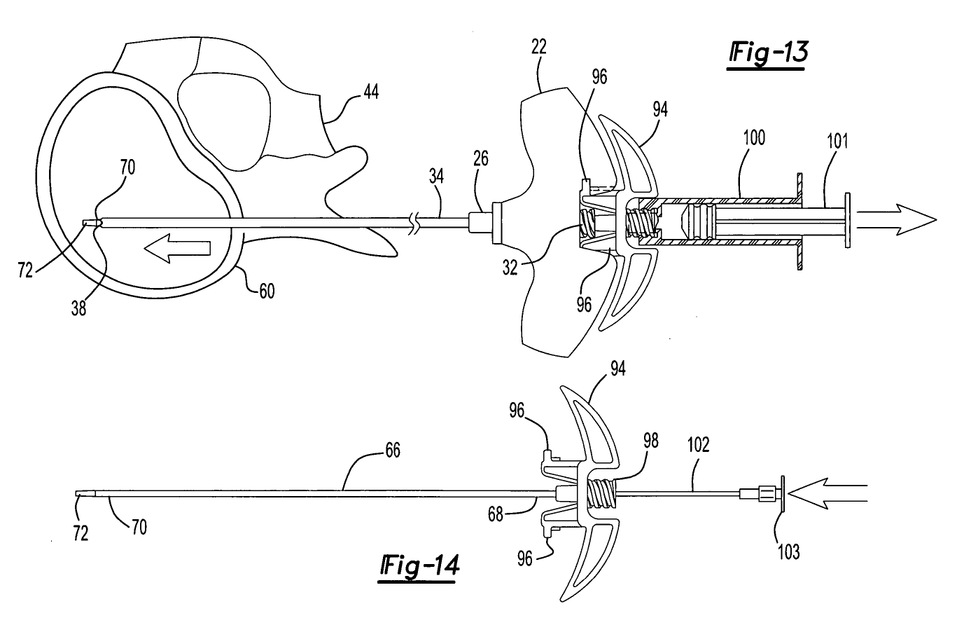

A tool handle 94 is connected to the proximal end 68 of the inner cannula 66. The tool handle 94 has an arcuate shape that fits comfortably and interchangeably within the palm of either hand of a user. The handle 94 is also specifically designed for interlocking engagement with the cannula handle 22, which maintains the inner cannula 66 in a stationary position relative to the access cannula 34. As is best shown in FIGS. 9 and 10, the tool handle 94 includes tongues 96 that are selectively inserted into the grooves 52 in the cannula handle 22 after the inner cannula 66 has been inserted through the access cannula 34 by rotating the tool handle 94 relative to the cannula handle 22 in the direction “D” shown. Interlocking the handles 94 and 22 in this manner enhances a user's ability to simultaneously rotate the coaxially positioned cannulas 66 and 34 and manipulate the same to advance the cutting edge 88 into the mass 44.

The tool handle 94 also includes a luer connector 98. The assembly 20 features a syringe 100, which is connected to the luer connector 98 and used to apply vacuum to the inner cannula 66, which in turn helps retain the biopsy specimen 74 within the swaged portion 72. The syringe 100 includes a plunger 101. The vacuum is applied by withdrawing the plunger 101 from the syringe 100. As is best shown in FIGS. 1, 4 and 17, the inner cannula 66 is withdrawn from the access cannula 34, and an obturator 102 with a cap 103 disposed on an end thereof is inserted through the inner cannula 66 to remove the biopsy specimen 74 therefrom by advancing the specimen 74 through the biopsy opening 80.

Once the inner cannula 66 has been removed, the access cannula 34 may remain in place within the bone mass 44 to maintain an access pathway to the harvest site 46. This permits other tools, diagnostic instruments and devices to be coupled to the access cannula 34 for performing additional procedures or delivering biomaterials, pharmaceuticals or other therapeutic agents to the harvest site 46. For example, a bone cement dispenser may be coupled to the male screw threads 32 for dispensing bone cement through the access cannula 34.

The subject invention also includes a method of collecting the biopsy specimen 74 of the bone mass 44. The method includes the step of inserting the introduction stylet 40 through the access cannula 34 to close the open end 38 of the access cannula 34. The stylet 40 and access cannula 34 are then inserted into the bone 44 to establish the harvest site 46. The introduction stylet 40 is removed from the access cannula 34, whereby the access cannula 34 maintains the access pathway to the harvest site 46.

The method continues by the portion 72 of the inner cannula 66 to form the non-deformable sidewall 76 having the exterior surface and the frustoconical interior surface 78, which is to extend radially inwardly at a predetermined angle “θ₁” from the distal end 70 to converge at the continuous annular cutting edge 84 defining the biopsy opening 80. The inner cannula 66 is inserted into the access cannula 34 until the cutting edge 34 extends through the open end 38. The access and inner cannulas 34 and 66, respectively, are advanced a predetermined distance into the bone 44, whereby the cutting edge 84 severs the specimen 74 from the bone 44 and urges the specimen 74 through the biopsy opening 80 into the inner cannula 66.

The final steps are removing the inner cannula 66 from the bone 44 and then removing the specimen 74 from the inner cannula 66.

The method is further defined as forming the pair of beveled facets 86 on the sidewall 86 of the inner cannula 66 to extend between the exterior surface 82 and the cutting edge 84 for rendering the cutting edge 84 sharp. Another step involves converging the facets 86 at the cutting edge 84 to define the pair of cusps 88. Still another step includes connecting the tool handle 94 to the proximal end 68 of the inner cannula 66, and forming a plurality of tongues 96 on tool handle 94 to extend therefrom.

The method is further defined by inserting the inner cannula 66 through the passageway 24 of the cannula handle 22 carried by the access cannula 34. The tool handle 94 is then rotated relative to the cannula handle 22 to engage the tongues 96 with the complementary grooves 52 on the cannula handle 22 to thereby retain the inner cannula 66 within the access cannula 34. Still another step involves connecting the syringe 100 to the luer connector 98 of the tool handle 94 and applying the vacuum to the inner cannula 34 for retaining the biopsy specimen 74 in the inner cannula 34.

Obviously, many modifications and variations of the present invention are possible in light of the teachings set forth above. The invention may be practiced other than as specifically described within the scope of the claims. Furthermore, the foregoing description of the preferred embodiment of the invention and the best mode for practicing the invention are provided for the purpose of illustration only and not for the purpose of limitation the invention being defined by the claims.

What is claimed is:

1. A biopsy assembly (20) for insertion into a mass of bone (44) to collect a biopsy specimen (74) of tissue therefrom, comprising:
a cannula handle (22) having a passageway (24) extending therethrough from a support end (26) to a receiving end (28) defining a recess (30) disposed about said passageway (24);
an access cannula (34) having a proximal end (36) supported in said passageway (24) and extending therefrom to an open end (38);
an introduction stylet (40) for selective insertion into said passageway (24) and through said access cannula (34) for advancing said access cannula (34) into the mass of bone (44) to establish a biopsy harvest site (46);
a cap (42) supporting said stylet (40); and
an inner cannula (66) for selective insertion through said passageway (24) into said access cannula (34) and extending from a proximal end (68) to a distal end (70);
said inner cannula (66) including a swaged portion (72) extending from said distal end (70) for retaining a biopsy specimen (74) therein and having a non-deformable sidewall (76) with a frustoconical interior surface (78) extending radially inwardly at a predetermined angle from said distal end (70) to a biopsy opening (80) for receiving the biopsy specimen (74) therethrough.

2. A biopsy assembly (20) as set forth in claim 1 wherein said sidewall (76) includes an exterior surface (82) and said biopsy opening (80) includes an annular cutting edge (84) having a pair of beveled facets (86) extending between said exterior surface (82) and said edge (84) for rendering said edge (84) sharp.

3. A biopsy assembly (20) as set forth in claim 2 wherein said facets (86) converge at said cutting edge (84) to define a pair of opposed cusps (88) extending therefrom.

4. A biopsy assembly (20) as set forth in claim 1 wherein said predetermined angle comprises 2°.

5. A biopsy assembly (20) as set forth in claim 2 wherein said exterior surface (82) extends radially inwardly at a predetermined angle from said distal end to said cutting edge (84).

6. A biopsy assembly (20) as set forth in claim 5 wherein said predetermined angle of said exterior surface (82) comprises 2°.

7. A biopsy assembly (20) as set forth in claim 1 and including a tool handle (94) connected to said proximal end (68) of said inner cannula (66).

8. A biopsy assembly as set forth in claim 7 wherein said tool handle (94) includes a luer connector (98) surrounding said inner cannula (66).

9. A biopsy assembly (20) as set forth in claim 8 and including a syringe (100) for connection to said luer connector of said tool handle (94) for applying a vacuum to said inner cannula (66) for retaining the biopsy specimen (74) within said swaged portion (72).

10. A biopsy assembly (20) as set forth in claim 1 wherein said cannula handle (22) includes male luer threads (32) disposed in said recess (20) about said receiving end (28).

11. A biopsy assembly (20) as set forth in claim 10 wherein said cap (42) includes opposed fingers (48) for selectively engaging said male luer threads (32) upon rotation of said cap (42) relative to said handle (22).

12. A biopsy assembly (20) as set forth in claim 1 wherein said cannula handle (22) and said cap (42) present a tongue and groove connection (50) for engaging upon rotation of said cap (42) relative to said handle (22).

13. A biopsy assembly (20) as set forth in claim 12 wherein said tongue and groove connection (50) includes a plurality of grooves (52) extending radially into said cannula handle (22) in said recess (30) and a plurality of tongues (54) extending radially from said cap (42) for engaging said grooves (52) upon rotation of said cap (42) relative to said handle (22).

14. A biopsy assembly (20) as set forth in claim 13 wherein said tool handle (94) includes tongues (96) for insertion into said grooves (52) in said cannula handle (22) upon insertion of said inner cannula (66) through said access cannula (34).

15. A biopsy assembly (20) as set forth in claim 1 and including an obturator (102) for selective insertion through said inner cannula (66) to remove the biopsy specimen (74) therefrom.

16. A biopsy assembly (20) for insertion into a mass of bone (44) to harvest a biopsy specimen (74) of tissue therefrom, said assembly (20) comprising; a cannula handle (22) having a passageway (24) extending therethrough from a support end (26) to a receiving end (28) defining a recess (30) disposed about said passageway (24) and including male luer threads (32) disposed in said recess (30) about said passageway (24);
an access cannula (34) supported in said passageway (24) and extending from said cannula handle (22) to an open end (38);
an introduction stylet (40) for selective insertion into said passageway (24) and through said access cannula (34) for closing said open end (38) thereof and for advancing said access cannula (34) into the mass of bone (44) to establish a biopsy harvest site (46);
a cap (42) supporting said stylet (40) and including opposed fingers (48) for engaging said male luer threads (32);
said cannula handle (22) and said cap (42) presenting a tongue and groove connection (50) for engaging upon rotation of said cap (42) relative to said handle (22) and including a plurality of grooves (52) extending radially into said cannula handle (22) in said recess (30) and a plurality of tongues (54) extending radially from said cap (42) for engaging said grooves (52) upon rotation of said cap (42) relative to said cannula handle (22);
an inner cannula (66) for selective insertion through said passageway (24) into said access cannula (34) and extending from a proximal end (68) to a distal end (70);
said inner cannula (66) including a swaged portion (72) extending from said distal end (70) for retaining a biopsy specimen (74) therein and having a non-deformable sidewall (76) including an exterior surface (82) and an interior surface (78) extending radially inwardly at a predetermined angle from said distal end (70) to a biopsy opening (80) having an annular cutting edge (84) and a pair of beveled facets (86) extending from said exterior surface (82) and converging at said cutting edge (84) to define a pair of opposed cusps (88) extending therefrom;
a tool handle (94) connected to said proximal end (68) of said inner cannula (66) and including tongues (96) for insertion into said grooves (52) in said cannula handle (22) upon insertion of said inner cannula (66) through said distal end (70) of said access cannula and having a luer connector (98) disposed about said proximal end (68);
a syringe (100) for selective connection to said luer connector (98) of said tool handle (94) for applying a vacuum to said inner cannula (66) for retaining the biopsy specimen (74) within said swaged portion (72); and
an obturator (102) for insertion through said inner cannula (66) to remove the biopsy specimen (74) therefrom.

17. A method of collecting a biopsy specimen of tissue (74) from a bone (44), comprising the steps of;
inserting an introduction stylet (40) through an access cannula (34) to close an open end (38) of the access cannula (34),
inserting the stylet (40) and access cannula (34) into a bone (44) to establish a harvest site (46) therein,
removing the introduction stylet (40) from the access cannula (34) whereby the access cannula (34) maintains an access pathway to the harvest site (46);
providing an inner cannula (66) extending from a proximal end (68) to a distal end (70);
swaging a portion (72) of the inner cannula (66) to form a non-deformable sidewall (76) having an exterior surface (82) and a frustoconical interior surface (79) extending radially inwardly at a predetermined angle from the distal end (70) to converge at a continuous annular cutting edge (84) defining a biopsy opening (80);
inserting the inner cannula (66) into the access cannula (34) and extending the cutting edge (78) through the open end (38),
advancing the access and inner cannulas (34), (66) a predetermined distance into the bone (44) whereby the cutting edge (84) severs a specimen (34) from the bone (44) and urges the specimen (74) through the biopsy opening (80) into the inner cannula (66),
removing the inner cannula (66) from the bone (44), and
removing the specimen (74) from the inner cannula (66).

18. A method as set forth in claim 17 further defined as forming a pair of beveled facets (86) on the sidewall (76) of the inner cannula (66) extending between the exterior surface (82) and the cutting edge (84) for rendering the cutting edge (84) sharp.

19. A method as set forth in claim 18 further defined as converging the facets (86) at the cutting edge (84) to define a pair of opposed cusps (88).

20. A method as set forth in claim 17 further defined as connecting a tool handle (94) to the proximal end (68) of the inner cannula (66).

21. A method as set forth in claim 20 further defined as forming a plurality of tongues (96) extending from the tool handle (94).

22. A method as set forth in claim 21 further defined as inserting the inner cannula (66) through a passageway (24) of a cannula handle (22) carried by the access cannula (34).

23. A method as set forth in claim 22 further defined as rotating the tool handle (94) relative to the cannula handle (22) to engage the tongues (96) with complementary grooves (52) in the cannula handle (22) to thereby retain the inner cannula (66) within the access cannula (39).

24. A method as set forth in claim 23 further defined as connecting a syringe (100) to a luer connector (98) of the tool handle (94) and applying a vacuum to the inner cannula (66) for retaining the biopsy specimen (74) in the inner cannula (66).
